(19) United States
(12) Patent Application Publication
(10) Pub. No.: US 2023/0270583 A1
(43) Pub. Date: Aug. 31, 2023

(54) DISPOSABLE MALE HYGIENE PRODUCT FOR A USER DURING URINATION

(71) Applicant: Vihang Mahesh GOSAVI, Mumbai (IN)

(72) Inventor: Vihang Mahesh GOSAVI, Mumbai (IN)

(21) Appl. No.: 18/049,850

(22) PCT Filed: Apr. 13, 2021
(86) PCT No.: PCT/IN2021/050368

§ 371 Date: Oct. 26, 2022

(30) Foreign Application Priority Data
Apr. 28, 2020 (IN) 202021018039

(51) Int. Cl. A61F 5/453; A61F 13/471

(52) U.S. Cl. CPC A61F 5/453; A61F 13/471

(57) ABSTRACT

The present invention discloses a disposable male hygiene product for a user during urination includes an elongated strip bend at a middle position to form two side arms, a non-separable pocket at an outer portion of an open end of each of the two side arms to insert at least two fingers or a thumb and a finger inside the non-separable pocket of each of the two side arms, wherein the disposable hygiene product is made of a liquid absorbing material that absorbs urine residue after the urination and provides a human contactless support to a penis during urination when a user holds the penis using the disposable hygiene product by inserting at least two fingers or a thumb and a finger inside the non-separable pocket of the two side arms.

### CROSS-REFERENCE TO RELATED APPLICATIONS

This application claims priority to International Patent Application Serial Number PCT/IN2021/050368, filed on Apr. 13, 2021, and published as WO2021220291A1, which claims priority to Indian Patent Application Serial Number IN202021018039, filed Apr. 28, 2020, the entirety of which are incorporated by reference.

### FIELD OF THE INVENTION

The present invention describes a product that is used to hold a penis to ensure male hygiene and cleanliness during urination and, more particularly, to the product which is made of a liquid absorbing material that absorbs excessive urine drops left. The present invention provides the product for a human contactless support to the penis during urination by inserting two fingers or one thumb and one finger inside the product and then holds the penis using the product. A user also uses the product to wipe out the urine drops or urine moisture after urination. The product of the present invention is easy to manufacture and lowers the cost of providing hygiene.

### BACKGROUND OF THE INVENTION

While urinating, a male stands and urinates into urinals or commodes by holding his penis with one hand while face various inconveniences such as dirty hand, urine droplets left after urination on a penis that causes his underwear or pants dirty, or urine may splash to the toilet seat, floor or on the user himself which leads to contact and transmission of germs. Dirty hands and clothes provide a favorable environment for the bacteria and viruses to grow which may lead to Urinary tract Infection and other infections. Hands should be clean before and after the urination, however, a problem arises when any means to clean the hands is not available.

Also, after urination, some drops of the urine or moisture remain at the glans of the penis which leads the underwear/pants dirty, causes skin rashes, and other health problems. Good genital hygiene and bathroom habits are the keys to avoid the introduction of harmful bacteria into the urinary tract or to avoid skin infections. Urine splash on clothes and body is another problem that a male faces in general. This problem is caused by factors including height from the toilet/urinary bowl and the angle of attack which is explained by many researchers. On one side where the researcher's findings include the suggestions on the penis holding angles during urination, on the other side it is important that holding of the penis should be comfortable and hygienic.

Several patents disclose the male hygienic products to be used during urination. One patent application US 2012/0087972 A1 describes “Winnie wipes” which is used to remove urine from the end/tip of a male's penis by dabbing, pressing, and wiping, with the “front” lightly moistened anti-bacterial liquid of the napkin\tissue side several times against the end/tip of his penis.

Another patent application KR 100958074B 1 describes “how to prepare the tissue for male urine” which discloses a method for manufacturing the toilet paper for the male urine comprises: a step of putting the penis into a hole which is bored in the fixed location of the center area of toilet paper; a step of surrounding the penis with the toilet paper; a step of urinating while the finger does not come in contact with the penis; and a step of wiping the urine drop and the urine moisture using the toilet paper which embraces the penis. Another patent application U.S. Pat. No. 4,064,880 A describes “Sanitary tubular napkin for males” which discloses a sanitary tubular napkin for male hygiene, formed into a set of napkins that are joined together; and the set of napkins is placed in a dispenser. A separating means permits the removal of one napkin and positions the subsequent napkin in a position for subsequent removal. However, the aforesaid mentioned patents or patent applications are not easy to use and manufacture. Therefore, the present invention provides an improved product to hold/cover the penis during urination for hygiene and cleanliness which is easy to adapt by the user. The present invention provides a better and comfortable holding/grip of the penis while keeping the user hands clean throughout its use. Also, the present invention is easy to manufacture, cost-effective and can be mass-produced using automated machines.

### OBJECTIVES OF THE INVENTION

The prime objective of the invention is to provide a product that eliminates direct contact/touch to the penis to ensure hygiene and cleanliness during urination. The present invention keeps the hands clean and eliminates the use of washing hands before or after urination.

Another objective of the invention is to provide the product to wipe the excessive urine drops left after urination or to clean the penis whenever required. This keeps the penis, undergarments or pants clean and avoids growing the bacteria or coming in contact with any virus. Another objective of the invention is to provide the product which is used by inserting either a user's two fingers or thumb and finger inside the product and thus, helps the user keep the hands clean throughout without touching the penis.

Another objective of the invention is to provide the product to hold/cover the penis comfortably to make the grip during urination and that to avoid the urine splashes anywhere.

Another objective of the invention is to provide the product to sanitize the penis with the sterilizing properties of the product. Another objective of the invention is to provide the product which is easily adaptable and convenient to use for the user. Another objective of the invention is to provide the product, which is easy to manufacture, store, transport, and cost-effective. Another objective of the invention is to provide the product to use in the Industry where hands hygiene is a concern and to hold the various items.

These and other objectives of the present invention will be apparent from the drawings and descriptions herein. Every object of the invention is attained by at least one embodiment of the invention. However, no embodiment necessarily meets every object set forth herein.

### SUMMARY OF THE INVENTION

One aspect of the present invention describes a disposable male hygiene product for a user during urination includes an elongated strip bend at a middle position to form two side arms, a non-separable pocket at an outer portion of an open end of each of the two side arms to insert at least two fingers or a thumb and a finger inside the non-separable pocket of each of the two side arms, wherein the disposable hygiene product is made of a liquid absorbing material that absorbs urine residue after the urination and provides a human contactless support to a penis during urination when a user holds the penis using the disposable hygiene product by inserting at least two fingers or a thumb and a finger inside the non-separable pocket of the two side arms.

Another aspect of the present invention describes a disposable male hygiene product includes an elongated strip bend at a middle position to form two side arms, a non-separable pocket at an outer portion of an open end of each of the two side arms to insert at least two fingers or a thumb and a finger inside the non-separable pocket of each of the two side arms and an elastic strip arranged around the rims of the pocket for better fitting as well as to support different finger thicknesses. Another aspect of the present invention describes a disposable male hygiene product includes an elongated strip bend at a middle position to form two side arms, a non-separable pocket at an outer portion of an open end of each of the two side arms to insert at least two fingers or a thumb and a finger inside the non-separable pocket of each of the two side arms and water-resistant layer either arranged as a top layer or an outer layer. It avoids accidental leaks to keep the hands dry during the process of wiping.

### BRIEF DESCRIPTION OF THE DRAWINGS

The drawings described herein are for illustrative purposes only of selected embodiments and not all possible implementations and are not intended to limit the scope of the present disclosure.

FIG. 1 represents the present disposable male hygienic product for a user during urination to maintain the proper hygiene standards.

FIGS. 2a to 2l represent the steps to form the present disposable male hygienic product with non-separable pockets for the insertion of a thumb and index finger of the user.

FIG. 3 represents one variation of FIG. 1 product as described.

FIG. 4 represents another variation of FIGS. 1 and 3 as described.

FIG. 5 represents another variation of FIGS. 1, 3, and 4 as described.

### DETAILED DESCRIPTION OF THE INVENTION

The following is a detailed description of the exemplary embodiments to illustrate the principles of the invention. The embodiments are provided to illustrate aspects of the invention, but the invention is not limited to any embodiment. The scope of the present invention encompasses numerous alternatives, modifications, and equivalent; it is limited only by the claims. Numerous specific details are outlined in the following description to provide a thorough understanding of the invention. However, the invention may be practiced according to the claims without some or all of these specific details.

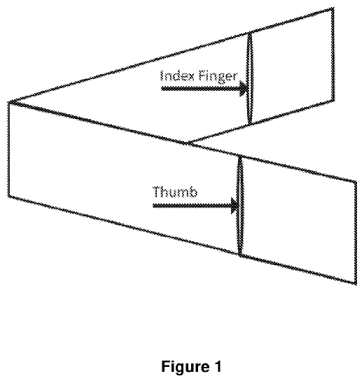

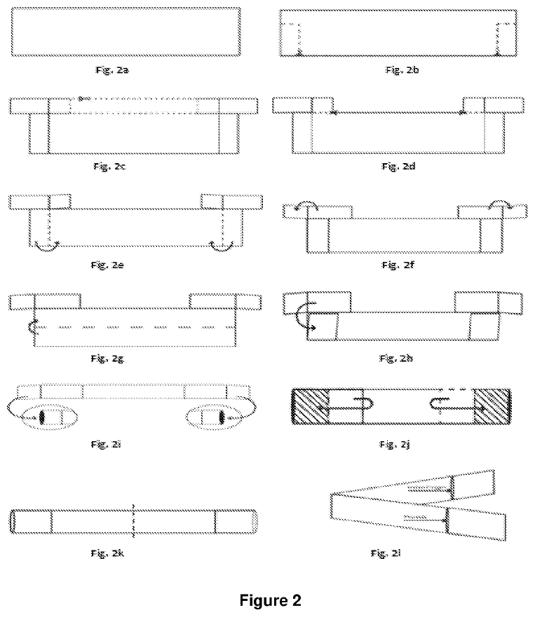

In reference of FIG. 1, one embodiment of the present invention provides a disposable male hygiene product for a user to use during urination that includes an elongated strip bend at a middle position to form two side arms, a non-separable pocket at an outer portion of an open end of each of the two side arms to insert at least two fingers or a thumb and a finger inside the non-separable pocket of each of the two side arms, wherein the disposable hygiene product is made of a liquid absorbing material that absorbs urine residue after the urination and provides a human contactless support to a penis during urination when a user holds the penis using the disposable hygiene product by inserting at least two fingers or a thumb and a finger inside the non-separable pocket of the two side arms. The formation includes various cuts and folds which are specific to provide the non-separable pockets as shown in FIG. 2 as an exemplary.

Preferably, the liquid absorbing material is a tissue paper or wipe which may vary in thickness. The absorbing material is selected from biodegradable material, non-biodegradable material, recycled paper or non-recycled paper based on industry availability.

The present invention has an impact on productivity in the industry. The machines and plants for the same can be installed for a large scale to provide high productivity in a short time. However, the product is also handmade and can be produced on a small scale. The present invention has a major advantage to be used in the medical industry for the urine samples. Tests can be done to detect the diseases or urine infections using the drops of urine absorbed by the disposable male hygienic product during the use by the user.

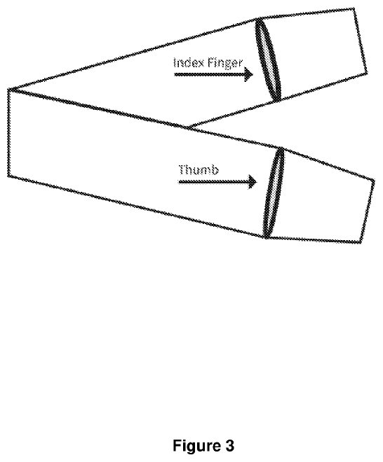

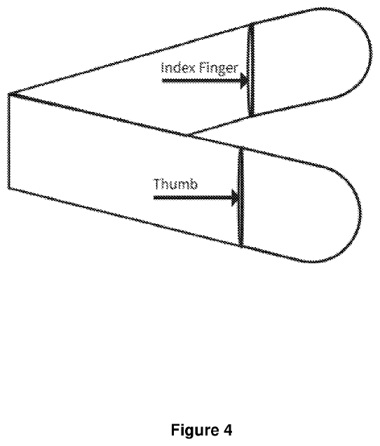

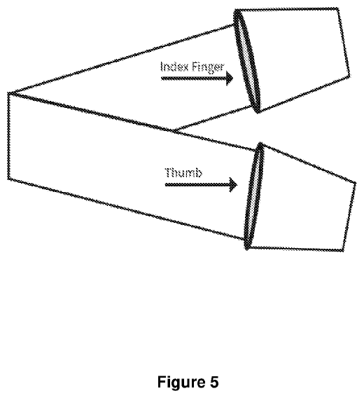

Additionally, the product either comprises a single layer or multiple layers of the absorbing material attach such that to act as a single layer. Further, an additional layer of a sterilizing agent is added to the absorbing material to sanitize the penis from contamination presence while wiping the penis after urination. The sterilizing agent is selected from the groups of the compound, which are available conventionally. The non-separable pocket is a square-shaped pocket as shown in FIG. 1. The non-separable pocket can be made in different shapes and sizes. In reference to FIG. 4, the shape of the non-separable pocket is U-shaped at the open end of each of the two side arms. In reference to FIGS. 3 and 5, the shape of the non-separable pocket is trapezoidal-shaped at the open end of each of the two side arms. In another example, the non-separable pocket is a V-shaped pocket having the V-shape at the open end of each of the two side arms. These are the examples, but are not limited to the shapes illustrated in the figures of the present invention.

In another embodiment, the non-separable pocket comprises a rough texture from an internal surface to provide a grip to hold the disposable male hygiene product by the user. This rough texture either is formed on the absorbing material or may be created later on the final product.

In another embodiment, the disposable male hygiene product comprises a groove in an open side of each of the non-separable pockets or at the ends of the products. In another variation, the disposable male hygiene product has a finger/thumb expression inside the area of the non-separable pocket to provide a better and comfortable grip. Further, the shape of the product is a concave shape to allow better ergonomic fitting for the fingers. Also, the product is chemically treated which is used to detect the color change when in contact with urine, this ensures that a used product is not used again by the user accidentally. The product may also have patterns, drawings, or text written over it.

In another embodiment, the disposable male hygiene product includes an elongated strip bend at a middle position to form two side arms, a non-separable pocket at an outer portion of an open end of each of the two side arms to insert at least two fingers or a thumb and a finger inside the non-separable pocket of each of the two side arms, and an elastic strip arranged around the rims of the pocket for better fitting as well as to support different finger thicknesses.

In another embodiment, the disposable male hygiene product includes an elongated strip bend at a middle position to form two side arms, a non-separable pocket at an outer portion of an open end of each of the two side arms to insert at least two fingers or a thumb and a finger inside the non-separable pocket of each of the two side arms, and water-resistant layer either arranged as a top layer or an outer layer. It avoids accidental leaks to keep the hands dry during the process of wiping. In a variation of any of the above-mentioned embodiments, the product of the present invention comprises a u-shaped material, preferably made of plastic which is attached to the bent side of the product or to the ends of the non-separable pocket to keep the product holding its shape.

In reference of FIGS. 2a to 2l represent the steps of the cuts and folds to form the present disposable male hygienic product manually with non-separable pockets for the insertion of fingers or a thumb and a finger by the user: FIG. 2a: Take a piece of liquid absorbing material, preferably in a rectangular shape as shown in the figure.

FIG. 2b: Cut along dotted lines forming a broad T-like shape.

FIG. 2c: Crease and fold the thin lines and cut the dotted lines. FIG. 2d:

Further cut the dotted lines as shown in the figure.

FIG. 2e: Folding inwards as arrows represented in the figure.

FIG. 2f: Flip the flap on the opposite side outwards.

FIG. 2g: Flip it over and fold the bottom third.

FIG. 2h: Fold the upper part. FIG. 2i: Fold the lips in lock towards both the sides.

FIG. 2j: Fold the dotted region of the top flap inside the shaded flap from both sides. FIG. 2k: Bend from the middle to get the final product.

FIG. 2l: Final Product to act as a disposable male hygiene article. Further, the present invention provides a method to use a male hygienic product for the use by the user during urination comprising the steps of:
1. access a disposable male hygienic product from a dispenser through one hand,
2. holding the product by inserting the fingers or a thumb and a finger in the non-separable pockets of the product, 3. holding the penis with the product,
4. dabbing/pressing or wiping the penis with the product, preferably holding through the thumb and the index finger, and
5. disposing of the product after use. In the preferred embodiment described in FIG. 1, the product is accessed from the dispenser which is fitted on the walls of the washrooms/restrooms. The product is stacked in plurality in the dispenser in such a way that the product is easily taken out using the fingers. Further, the product can be stacked in a medium of storage or a tray to be accessed by the user.

The advantages and uses of the present invention are further extended to the pathology labs or science labs for the handling of scientific and medical equipment and samples, in hospitals, in archeological surveys for precaution while handling a specimen, in the jewelry industry while working and by tattoo artists during tattoo making. These are only some examples of uses and can be used in other industries as well where hands or products need to keep clean and contamination-free to act as hand gloves.

Although preferred embodiments of the invention have been illustrated and described, it will at once be apparent to those skilled in the art that the invention includes advantages and features over and beyond the specific illustrated construction. Accordingly, it is intended that the scope of the invention be limited solely by the scope of the hereinafter appended claims, and not by the foregoing specification, when interpreted in light of the relevant prior art.

The foregoing method descriptions and diagrams/figures are provided merely as illustrative examples and are not intended to require or imply that the operations of various aspects must be performed in the order presented. As will be appreciated by one of skill in the art, the order of operations in the aspects described herein may be performed in any order. Words such as “thereafter,” “then,” “next,” etc. are not intended to limit the order of the operations; such words are used to guide the reader through the description of the methods and systems described herein. Further, any reference to claim elements in the singular, for example, using the articles “a,” “an,” or “the” is not to be construed as limiting the element to the singular.

The preceding description of the disclosed aspects is provided to enable any person skilled in the art to make, implement, or use the claims. Various modifications to these aspects will be readily apparent to those skilled in the art, and the generic principles defined herein may be applied to other aspects without departing from the scope of the claims. Thus, the present disclosure is not intended to be limited to the aspects illustrated herein but is to be accorded the widest scope consistent with the claims disclosed herein.

We claim:

1. A disposable hygiene product for a user during urination, comprising:
an elongated strip bend at a middle position to form two side arms;
a non-separable pocket at an outer portion of an open end of each of the two side arms to insert at least two fingers inside the non-separable pocket of each of the two side arms, wherein the disposable hygiene product is made of a liquid absorbing material that absorbs urine residue after the urination and provides a human contactless support to a penis during urination when a user holds the penis using the disposable hygiene product by inserting the two fingers inside the non-separable pocket of the two side arms.

2. The disposable hygiene product as claimed in claim 1, wherein the liquid absorbing material is a single layer paper.

3. The disposable hygiene product as claimed in claim 1, wherein the liquid absorbing material is a multilayer paper.

4. The disposable hygiene product as claimed in claim 1, wherein the liquid absorbing material comprises a sterilizing layer.

5. The disposable hygiene product as claimed in claim 1, wherein an inner side of the product comprises the absorbing material to provide support to hold the penis and wipe off the extra urine drops, and an outer side of the product comprises a water-resistant layer to keep the hands clean from the wet absorbing layer.

6. The disposable hygiene product as claimed in claim 1, wherein the non-separable pocket is a U-shaped pocket having the U-shape at the open end of each of the two side arms.

7. The disposable hygiene product as claimed in claim 1, wherein the non-separable pocket is a square pocket.

8. The disposable hygiene product as claimed in claim 1, wherein the non-separable pocket is a V-shaped pocket having the V-shape at the open end of each of the two side arms.

9. The disposable hygiene product as claimed in claim 1, wherein the non-separable pocket is a trapezoidal shape at the open end of each of the two side arms.

10. The disposable hygiene product as claimed in claim 1, wherein an internal surface of non-separable pocket has a rough texture to provide a grip.

11. The disposable hygiene product as claimed in claim 5, wherein a surface of the inner side of non-separable pocket comprises grooves to provide a grip.

12. The disposable hygiene product as claimed in claim 5, wherein a surface of the outer side of non-separable pocket comprises grooves to provide a grip.

13. The disposable hygiene product as claimed in claim 1, wherein the non-separable pocket comprises an elastic strip around the rims.

14. The disposable hygiene product as claimed in claim 1, wherein the product is used in at least one of a pathology lab or a science lab for the handling of scientific and medical equipment, and samples, hospitals, archeological surveys for precaution while handling a specimen, jewelry industry, or tattoo artists.
